(19) United States
(12) Patent Application Publication
(10) Pub. No.: US 2008/0118914 A1
(43) Pub. Date: May 22, 2008

(54) Follistatin gene as a genetic marker for first parity litter size in pigs

(76) Inventor: Charlotte Dawn Blowe, Raleigh, NC (US)

Correspondence Address: Charlotte Dawn Blowe, 204 Essex Lane S., Clayton, NC, 27520, US

(21) Appl. No.: 11/324,525

(22) Filed: Jan. 4, 2006

(51) Int. Cl. C12Q 1/68; C07H 21/04

(52) U.S. Cl. 435/6; 536/23.1

(57) ABSTRACT

Disclosed herein are genetic markers for litter size in first parity gilts, methods for identifying such markers, and methods of screening pigs to determine those more or less likely to have large litters in their first parity and more or less likely to produce litters with offspring who also posses that same genetic ability to have larger first parity litters and preferably selecting those pigs for future breeding purposes. The markers are based upon the presence or absence of certain polymorphisms in the pig follistatin gene.

### CROSS-REFERENCE TO RELATED APPLICATIONS

Current application claims priority of provisional patent No. 60/640,314

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISC APPENDIX

Not applicable.

### BACKGROUND OF THE INVENTION

This invention relates generally to the detection of genetic differences for increased first parity litter size among pigs and particularly use of a genetic marker in the follistatin gene, found on pig chromosome 16, which is indicative of the heritable trait of litter size in pigs.

Reproductive success is dependent upon a number of complex physiological events in both sexes. If genetic markers can be found which are linked to quantitative trait loci (QTL) affecting economic traits in swine, these markers could be used to increase selection accuracy.

The ability to pursue a specific beneficial genetic allele involves the identification of a DNA molecular indicator or marker for a major effect gene. The marker may be linked to a single gene with a major effect or linked to a quantity of genes with additive effects. DNA markers have several advantages; segregation is easily gauged and is explicit, and DNA markers are co-dominant, i.e., heterozygous and homozygous animals are easily distinguished. Another advantage is an increase in selection accuracy due to addition of information directly related to genotype. Still another advantage is the possibility of reducing generation interval by allowing selection to be made at an earlier age because this technique is not age or sex dependent. Once a marker system is ascertained selection evaluations could be made straightforwardly, since DNA markers can be analyzed any time after a genetic sample can be gathered from the individual infant animal.

The use of genetic disparity in genes has become a useful marker system for selection in reproduction. For example U.S. Pat. No. 5,374,526 issued Dec. 20, 1994 to Rothschild discloses a polymorphism in the pig estrogen receptor gene which is coupled with an increase in litter size, the disclosure of which is incorporated herein by reference. U.S. Pat. No. 5,935,784 issued on Aug. 10, 1999 to Rothschild et al., incorporated herein by reference, disclosed polymorphic markers in the pig prolactin receptor gene which are linked with larger litter size and general reproductive competence.

The current invention provides a genetic marker, established upon the finding of a polymorphism in the follistatin gene, which relates to first parity litter size in pigs. This will permit genetic typing of pigs for their follistatin genes and for determination of the relationship of specific RFLPs to litter size. It will also permit the identification of individual males and females that carry the gene for litter size. Thus, the markers may be selection tools in breeding programs to develop lines and breeds that produce litters containing offspring with more desirable litter sizes.

According to the invention two polymorphisms were identified in the follistatin gene which are associated with larger first parity litter size.

### BRIEF SUMMARY OF THE INVENTION

To achieve the objects and in agreement with the purpose of the invention, as embodied and generally described herein, the present invention provides a process for screening pigs to determine those which will be likely to produce larger litters and offspring with the ability to produce larger litters when bred or to select against pigs which have alleles indicating the less favorable reproductive trait. Thus, the present invention provides a method for screening pigs to determine those more likely to produce larger litters themselves and offspring which will also possess that same quality, and/or those less likely to produce small litters, which method comprises the steps 1) obtaining a sample of genomic DNA from a pig; and 2) analyzing the genomic DNA obtained in 1) to determine which follistatin allele(s) is/are present. Briefly, a sample of genetic material is obtained from a pig, and the sample is analyzed to determine the presence or absence of at least one of the polymorphisms in the follistatin gene that is correlated with increased first parity litter size.

In one embodiment the polymorphisms are restriction fragment length polymorphisms and the assay comprises identifying the pig follistatin gene from isolated pig genetic material; exposing the gene to a restriction endonuclease that yields restriction fragments of the gene of varying size; separating those restriction fragments to form a restriction pattern, such as by electrophoresis or HPLC separation; and contrasting the resulting restriction fragment pattern from a pig follistatin gene that is either known to have or not to have the desired marker. If a pig tests positive for the favorable marker, such pig can be considered for inclusion in the breeding program. If the pig does not test positive for the marker genotype the pig can be culled from the group and otherwise used.

In a most preferred embodiment the gene is isolated by the use of primers and DNA polymerase to amplify a specific region of the gene which contains one of the polymorphisms. Then the amplified region is digested with a restriction endonuclease and fragments are separated. Visualization of the RFLP pattern is by straightforward staining of the fragments, or by labeling the primers or the nucleoside triphosphates used in amplification.

It is also possible to ascertain linkage between precise alleles of other DNA markers and alleles of DNA markers known to be associated with a particular gene (e.g. the follistatin gene discussed herein), which have previously been found on the same chromosome. Thus, in the current condition, taking the follistatin gene, it would be possible, at least in the interim, to select for pigs expected to produce large litters, or otherwise against pigs likely to produce smaller litters, indirectly, by selecting for certain alleles of the follistatin associated marker through the selection of specific alleles of alternative chromosome 16 markers including S0111, S0006, S0077, S0390, S0326 and S0363.

The invention further comprises a kit for assessing a sample of pig DNA for the presence in pig genetic material of a preferred genetic marker located in the pig follistatin gene indicative of the heritable trait of litter size. At a minimum, the kit is a container with one or more reagents that identify a polymorphism either in or associated with the pig follistatin gene. Preferably, the reagent is a set of oligonucleotide primers capable of amplifying a fragment of the pig follistatin gene that contains one of the polymorphisms. The kit further may include a restriction enzyme that cleaves the pig follistatin gene in at least one place. In a most preferred embodiment the restriction enzyme is MspI or Fnu4HI or one which cuts at the same recognition sites.

The accompanying figures, which are incorporated herein and which constitute a part of this specification, illustrate one embodiment of the invention and, together with the description, serve to explain the principles of the invention.

It is an object of the invention to provide a method of screening pigs to determine those more likely to produce offspring containing this desirable reproductive trait.

Another object of the invention is to provide a method for identifying genetic markers for pig litter size.

A further object of the invention is to provide genetic markers for selection and breeding to obtain pigs that will be expected to have this desirable reproductive trait.

Yet another object of the invention is to provide a kit for evaluating a sample of pig DNA for specific genetic markers for litter size.

Supplementary objects and benefits of the invention will be set forth in part in the description that follows, and in part will be obvious from the description, or may be learned by the practice of the invention. The objects and advantages of the invention will be attained by means of the instrumentalities and amalgamations predominantly pointed out in the appended claims.

### DETAILED DESCRIPTION OF THE INVENTION

Reference will now be made in detail to the currently preferred quintessence of the invention, which together with the following examples, serve to explain the principles of the invention.

The invention relates to genetic markers associated with litter size in pigs. It provides a method of screening pigs to determine those more likely to produce a larger litter and offspring with this favorable reproductive trait of larger litters when bred by identifying the presence or absence of a polymorphism in the follistatin gene that is correlated with litter size. Used herein, the term “increased litter size” means a biologically significant increase in litter size above the mean of a given population.

Thus, the invention correlates to genetic markers and techniques of recognizing those markers in a pig of a particular breed, strain, population, or group, whereby the selected pig is more likely to produce a litter that is significantly above the average reproductive performance for that particular breed, strain, population, or group. Any method of identifying the presence or absence of this marker may be used, including for example single-strand conformation polymorphism (SSCP) analysis, RFLP analysis, heteroduplex analysis, denaturing gradient gel electrophoresis, and temperature gradient electrophoresis, ligase chain reaction or even direct sequencing of the follistatin gene and examination for the MspI or Fnu4HI or other comparable enzymes' recognition pattern.

Other potential methods include non-gel systems such as TaqMan™ (Perkin Elmer). In this method oligonucleotide PCR primers are designed to border the mutation in question and allow amplification. A third oligonucleotide probe is then created to hybridize or bind to the region that contains the base found to be subject to change between different alleles of the follistatin gene. The probe is tagged with fluorescent dyes at both the 5′ and 3′ ends. The dyes are selected such that while in close proximity to each other the fluorescence of one of them is suppressed or quenched by the other and thus is undetectable. Extension carried out by Taq DNA polymerase from the 5′ primer leads to the excision of the attached dye at the 5′ end of the annealed probe as a result of the 5′ nuclease activity of the Taq DNA polymerase. This action removes the suppressing effect thus, allowing exposure of the fluorescence from the dye at the 3′ end of the probe. The discrimination between differing DNA sequences arises from the fact that if the binding of the probe to the template strand of DNA is incomplete, i.e. there is a mismatch of some sort, the cleavage of the dye does not occur. Thus only when the nucleotide sequence of the probe is entirely corresponding to the template molecule to which it is attached will quenching be eliminated. A reaction blend can contain two different probe sequences each one intended to detect different alleles that could be present thus allowing the exposure of both alleles in one reaction.

The use of RFLPs is the favored technique of detecting the polymorphisms. However, because the use of RFLP analysis relies ultimately on polymorphisms and DNA restriction sites along the nucleic acid molecule, other assays and techniques of detecting the polymorphisms can also be employed. Such procedures consist of ones that analyze the polymorphic gene product and identify polymorphisms by detecting the consequential distinctions in the gene product.

A RFLP analysis in general is a technique well-known to those skilled in the art. See, for example, U.S. Pat. No. 4,582,788 issued Apr. 15, 1986 to Erlich and U.S. Pat. No. 4,666,828 issued May 19, 1987 to Gusella, U.S. Pat. No. 4,772,549 issued Sep. 20, 1988 to Frossard, and U.S. Pat. No. 4,861,708 issued Aug. 29, 1989 to Frossard, all of which are incorporated herein by reference. Generally speaking, the technique requires obtaining the DNA to be studied, digesting the DNA with restriction endonucleases, separating the resulting fragments, and detecting the fragments of assorted genes.

In the present invention, a sample of genetic material is acquired from a pig. Samples can be obtained from blood, tissue, semen, hair, buccal swabs, saliva, etc. Generally, white blood cells are used as the source, and the genetic material is DNA. An adequate amount of cells are obtained to provide a sufficient amount of DNA for analysis. This amount will be known or readily determinable by those skilled in the art. The DNA is isolated from the blood cells or other suitable sample by techniques known to those skilled in the art.

Next the region containing the polymorphism is amplified by the use of primers and customary techniques, such as the polymerase chain reaction. This technique is described in U.S. Pat. No. 4,683,195, issued Jul. 28, 1987 to Mullis et al., U.S. Pat. No. 4,683,202, issued Jul. 28, 1987 to Mullis, U.S. Pat. No. 4,800,159 issued Jan. 24, 1989 to Mullis, et al., U.S. Pat. No. 4,889,818 issued Dec. 26, 1989 to Gelfand, et al., and U.S. Pat. No. 4,902,624, issued Feb. 20, 1990 to Clumbus, et al., all of which are incorporated herein by reference. The choice of primers is discussed in the references mentioned and incorporated herein. The primers should amplify the follistatin gene or at a minimum the portion of the follistatin gene which contains one of the polymorphic sites (or one associated with it) identified herein.

The amplified isolated DNA is then digested with a restriction endonuclease that cleaves or severs DNA hydrolytically at a restriction site which is a precise nucleotide sequence. These endonucleases, also referred to as restriction enzymes, are familiar to those trained in the art. For the present invention, a restriction enzyme should be chosen that cuts the pig follistatin gene in at least one location, creating at least a pair of gene fragments. An assessment is made as to whether or not any of the resulting fragments are polymorphic and if any particular polymorphism (RFLP) is linked with litter size by methods known in the art in combination with the knowledge enclosed herein. Preferably, the restriction enzyme is Msp I for the FS1 marker or Fnu4HI for the FS2 marker or other appropriate enzyme. The enzyme Msp I cleaves double stranded DNA at the sequence 5′ CCGG 3′, with the actual cut occurring between the pair of C's. The restriction enzyme Fnu4HI cuts at the recognition sequence 5′ GCNGC 3′ with the cut occurring between the C and N. The quantity of enzyme to be added to the sample containing the pig DNA and the other suitable reagents for treating the sample will be unhesitatingly ascertainable to persons skilled in the art, given the information contained herein.

The resulting restriction fragments are then investigated by known practices that usually entail either the separation of fragments and visualization by staining or subsequent blotting and hybridization to acquire a particular pattern or the resolution of varying sizes of fragments. The preferred separation method is gel electrophoresis.

In this process, the digested DNA fragments are separated in a supporting medium based on size using an electric current. Gel sheets, such as agarose or similar substance, are normally used as the supporting medium. The sample, containing the restriction fragments, is added to the negative end of the gel. At least one molecular marker of known size is run on the same gel as a control to allow a size estimation of the restriction fragments. This technique usually permits a measure of resolution that separates DNA restriction fragments that differ in size from each other by as few as 100 base pairs.

In other embodiments, the resulting restriction fragments are denatured and relocated actually from the gel to a solid support, such as a nylon membrane, by contacting the gel with the filter in the existence of suitable reagents and under appropriate circumstances that encourage the transfer of the DNA from the gel to the membrane. These reagents and conditions are well-known to those skilled in the art. The relative positions of the fragments resulting from the separation process are sustained.

The next step entails the recognition of the assorted categories of sizes of the fragments or, otherwise, the detection of a particular sized fragment. The second may be of particular significance because it is a genetic marker that is correlated with litter size. This is preferably achieved via staining the fragments with a chemical ethidium bromide.

Another procedure is the utilization of a hybridization probe. Such a probe is an oligonucleotide or polynucleotide that is adequately complementary or homologous to the fragments to bind with them, creating probe-fragment complexes. Preferably, the probe is a cDNA probe. The oligonucleotide or polynucleotide has a detectable entity attached to it that serves as a label. This allows the detection of the fragments, to which the probes are annealed. Probes are labeled by customary labeling methods, like a radiolabel, enzyme label, fluorescent label, biotin-avidin label, and the like. See U.S. Pat. No. 4,711,955 issued Dec. 8, 1987 to Ward et al. and U.S. Pat. No. 4,868,103 issued Sep. 19, 1989 to Stavrianopoulos et al., both of which are incorporated herein by reference.

The probes are placed in contact with the nylon membrane containing the restriction fragments for an ample length of time and under suitable hybridizing surroundings for the probes to attach to the fragments. The filter is then preferably rinsed to eliminate unattached probes and other unnecessary materials.

The probe-fragment complexes, which are hybridized to the filter, are then recognized by known methods. The fragments of interest are visualized according to the chosen detection method, which would be known to someone skilled in the art.

The recognition phase supplies a pattern, ensuing from the partitioning of the fragments based on size. Comparison of these fragments with molecular marker fragments of known size that have also been run on the same gel allows the approximation of size of the various fragments. The assorted polymorphisms in the pig follistatin gene are then determined by evaluation of the configurations created by similar assay of DNA from a number of different pigs. For some of the individual pigs, the patterns will vary from the normal pattern seen in most of the other pigs. This will be because of one or more restriction fragment length polymorphisms, i.e., restriction fragments of a varying length produced by the enzyme that cuts the pig follistatin gene. This illustrates different base pair sequences in such pigs.

Once a specific RFLP has been discovered, i.e., a restriction fragment of a particular length, a probe to this segment may be produced by the use of known methods. This allows different and quicker systems for detecting such polymorphism. For instance, once the DNA is digested, a sandwich hybridization format can be utilized. An assay of this type is disclosed in U.S. Pat. No. 4,486,539 issued Dec. 4, 1984 to Ranki, et al., and U.S. Pat. No. 4,563,419 issued Jan. 7, 1986 to Ranki, et al., both of which are incorporated herein by reference. The sample is brought into contact with a capture probe that is stationary on a solid carrier. The probe hybridizes the segment. Then the carrier is rinsed, and a labeled detection probe is added. After further rinsing, the detection probe is recognized, thus signifying the incidence of the preferred fragment.

In yet another embodiment, once the RFLP pattern has been determined or a specific polymorphic fragment has been discovered, it is compared to a second, fragment with a known RFLP pattern that is associated with litter size. This second fragment has also been determined from the pig follistatin gene, using the same restriction endonuclease as the first and the same probe or an equal thereof under the same circumstances.

In a different embodiment of the invention, the restriction fragments can be identified by solution hybridization. In this process, the restriction fragments are bound with the probe and then separated. The separated probe-fragment complexes are then visualized as discussed above. Generally, they are seen on the gel without transport to filter paper.

In a most favored embodiment the polymorphism is detected by PCR amplification minus a probe. This technique is known to those of skill in the art and is disclosed in U.S. Pat. No. 4,795,699 entitled “DNA Polymerase” and U.S. Pat. No. 4,965,188. “Process for Amplifying, Detecting, and/or Cloning Nucleic Sequences Using a Thermostable Enzyme” both of which are incorporated herein by reference.

For this technique primers are designed to amplify the portion of the gene in which the polymorphism lies. Therefore, primers, which are preferably 4-30 bases, are constructed established on the sequence flanking the polymorphism including a forward 5′, primer and a reverse or anti-sense 3′ primer. The primers do not have to be the exact complement, and considerably equivalent sequences are also adequate. A DNA polymerase, such as Taq polymerase (many such polymerases are recognized and commercially accessible), is then added in the presence of the four nucleoside triphosphates and frequently a buffering agent. Detection is made possible by straightforward staining, such as with ethidium bromide, of separated PCR products to detect for products of predicted sizes based on the length of the amplified region. Reaction times, reagents, and primer design are all known to those skilled in the art and are discussed in the patents incorporated herein by reference. Additional PCR amplification may be combined with Single Strand Confirmation Polymorphism (SSCP).

Even though the previously mentioned techniques are depicted in terms of utilizing a solitary restriction enzyme and a lone set of primers, the methods are not so restricted. One or more supplementary enzymes and/or probes and/or primers can be used, if preferred. Additional enzymes, constructed probes and primers can be discovered through routine experimentation that could be performed by one skilled in the art.

Pig litter size genetic markers are determined as follows. Individuals from both sexes of the same background, i.e. breed, breed cross, or derived from similar genetic lineages and grown under regular conditions, are chosen. The first parity litter size of each pig is determined. Restriction fragment length polymorphism analysis of the DNA is performed as discussed previously in order to determine polymorphisms in the follistatin gene of each pig. The polymorphisms are then related to the litter size of each individual animal. At least 20 and preferably a 100 pigs are used to make these determinations.

When this investigation is performed and the polymorphism(s) is/are determined by PCR-RFLP technique using the restriction endonuclease MspI, Fnu4HI or comparable enzyme and PCR primers may be created using corresponding known human follistatin sequences, or using pig follistatin gene sequence information as illustrated herein or even designed from sequences obtained using linkage data from nearby neighboring genes. According to the invention a primer set has been selected which amplifies a 625 bp fragment (forward primer 5′-GGACCGAGGAGGACGTAAAT-3′ (SEQ ID NO: 1) and the reverse primer 5′-GGCCTTTCCAGGTGATGTTA-3′ (SEQ ID NO: 2)). After PCR a restriction digest is performed and restriction polymorphic fragments of approximately 125, 200, 225, and 425 base pairs are generated. The fragment of size 425 bp is designated as the favorable B allele, while the A allele is identified by the presence of two fragments of size 220 bp and 225 bp. The 125 base pair fragment is a monomorphic band. Another set of primers was also created for a second polymorphism closely related to the first one. These primers are 5′-TGCCGAATGAACAAGAAGAA-3′, SEQ ID NO:3, and 5′-CAGAAAACATCCCGACAGGT-3′, SEQ ID NO:4, which produces a product of 450 bp. Upon digestion of the amplified fragment with Fnu4HI fragments of size 300, 200, 125 and 75 base pairs. The favorable B allele is identifiable by fragments of 125 bp and 75 bp in size, while the A allele is denoted by the presence of a 200 bp fragment. The 300 base pair fragment is a monomorphic fragment. The genotype associated with increased litter size is BB for either set of primers and enzymes.

The reagents appropriate for employing the techniques of the invention may be packaged into convenient kits. The kits supply the essential materials, packaged into proper containers. At a bare minimum, the kit includes a reagent that recognizes a polymorphism in the pig follistatin gene that is associated with litter size. Preferably, the reagent is a set of PCR reagents including a primer set, DNA polymerase, a suitable buffering agent, and 4 nucleoside triphosphates that bind with the pig follistatin gene or a portion thereof. Preferably, the PCR reagents and a restriction endonuclease that differentially cuts the pig follistatin gene in at least one place are included in the kit. In a predominantly preferred embodiment of the invention, the forward primer is SEQ ID NO:1 and the reverse primer is SEQ ID NO:2 and the restriction enzyme is Msp I, or the primer is SEQ ID NO:3 and 4 and the enzyme is Fnu4HI. Preferably, the kit further includes additional materials, such as a sample collection vessel, DNA extracting reagents, reagents for detecting or visualizing the polymorphism, and a control. Additional reagents employed for hybridization, prehybridization, etc. may also be incorporated, if desired.

The techniques and resources of the invention may also be utilized in a more general manner to evaluate pig DNA, genetically type individual animals, and detect genetic differences existing among pigs. Specifically, a sample of pig genomic DNA may be assessed by reference to single or multiple controls to determine if a polymorphism in the follistatin gene is present. Preferably, PCR-RFLP analysis is executed with respect to the pig follistatin gene, and the outcomes are contrasted with a control. The control is the result of a PCR-RFLP analysis of the swine follistatin gene of a different individual where the polymorphism of the pig follistatin gene is known. Similarly, the follistatin genotype of a pig may be deciphered by acquiring a sample of its genomic material, performing PCR-RFLP analysis of the follistatin gene in the DNA, and evaluating the results with a control. Again, the control is the result of PCR-RFLP analysis of the follistatin gene of a different pig. The results genetically type the pig by indicating the polymorphism in its follistatin genes. Lastly, genetic differences among pigs can be discovered by attaining samples of the genomic DNA from at least a pair of pigs, recognizing the presence or absence of a polymorphism in the follistatin gene, and evaluating the results.

These techniques are functional for identifying the genetic markers relating to litter size, as discussed above, for distinguishing other polymorphisms in the follistatin gene that may be associated with other characteristics, and for the general scientific analysis of pig genotypes and phenotypes.

The genetic markers, protocols, and kits of the invention are also valuable in a breeding program to advance litter size in a breed, line, or population of pigs. Unremitting selection and propagation of sows that are at least heterozygous and preferably homozygous for a polymorphism associated with litter size would lead to a breed, line, or population being more reproductively efficient. Hence, the markers are selection tools.

It is to be understood that the function of the teachings of the present invention to a definite dilemma or environment will be within the competence of one having ordinary skill in the art considering the information enclosed herein. The examples of the products and processes of the present invention emerge in the subsequent examples.

### EXAMPLE 1

Population Description: Direct selection for increased litter size was practiced for eleven generations in a Large White-Landrace composite population. Briefly, pigs were selected from the largest litters where litter size was based on number of fully formed pigs. In order to minimize maternal effects on litter size, litters were standardized at birth so that no replacement gilts were reared in a litter greater than 10 pigs. A control line was maintained using random selection. The following traits were recorded in generations ten and eleven: number of fully formed pigs at birth (NFF), number of pigs born alive (NBA) and number of mummified fetuses (MUM).

Sample Collection: Genomic DNA can be extracted from any biological source as long as said source contains cell nuclei, for example white blood cells, sperm cells, hair follicles, buccal swabs, ear notches, skin, muscle, etc. The techniques, which follow, deal with white blood cells and tissue samples.

Buffy coat (white blood cells): Several ten milliliter samples were collected from each individual in generation ten using vacutainers containing Tris EDTA. Blood samples were centrifuged at 4° C. for 20 minutes. The buffy coat was removed from each sample and placed into a labeled micro centrifuge tube. Blood samples were stored at −20° C.

Tissue: Tail tissue was collected from each individual in generation eleven at birth. Tissues were stored in labeled containers at −20° C. Any individual needing to be re-sampled had ear tissue collected. Ear tissue was treated in the same manner as tail tissue.

### EXAMPLE 2

DNA Extraction: There are various DNA extraction protocols available commercially and known to those skilled in the art. The techniques presented here are not the only method by which DNA can be extracted from a given sample type.

Buffy coat: Modifications were made to Gentra's (Minneapolis, Minn.) protocol for the PureGene™ extraction kit. A mixture of two parts distilled water and one part buffy coat was put through the modified protocol, which involved extended incubations and centrifugation steps. Red blood cells were lysed first. Then white blood cells were collected and lysed to release DNA. Samples were treated with RNAse A to degrade RNA. Proteins were then precipitated out of the samples. The DNA was washed and hydrated. Samples were stored at 4° C. Concentrations of each individual were determined. Any sample with a concentration less than 25 nano grams/micro liter was re-extracted.

Tissue: Modifications to the Gentra protocol were also necessary for DNA extraction from tissue samples. Tissue pieces weighting 0.005-0.01 g were minced and added to Cell Lysis Solution™. Protease was added to each tissue sample to degrade the proteins. Protein Precipitation Solution™ was added to each sample to separate proteins from DNA. Then DNA was precipitated with isopropanol and washed with 70% ethanol. After precipitation, DNA Hydrating Solution™ was added to each sample to re-suspend DNA. Samples were stored at 4° C. until further use. Concentrations were found and any samples needing re-extracting were run through this protocol again.

### EXAMPLE 3

Designing and Optimizing PCR Primers: The method of PCR amplification is widely recognized and known to those skilled in the art. This process can vary in many factors, including, but not limited to, primers, restriction enzymes, reaction conditions (times, temperatures, etc.) and reagents, all of which are intended to be included in this invention.

Designing and Choosing Primers: Expressed Tag Sequences (EST) for desired genes were found using the pig gene index on The Institute for Genomic Research's website (www.tigr.org). Then ESTs were “blated” against the Human Golden Pathway (http://genome.ucsc.edu). Regions of homology spanning non-homologous regions of one kilobase or less were chosen. Homologous regions were plugged into Primer3, a primer designing program (www.genome.wi.mit.edu/cgi-bin/primer/primer3_www.cgi) in order to look for possible primer pairs. Primer pairs were chosen based on whether they would span the non-homologous regions, which were likely to be introns and thus, more likely to contain polymorphisms. Primers chosen were:

Follistatin 1 (FS1) forward:
GGACCGAGGAGGACGTAAAT
reverse:
GGCCTTTCCAGGTGATGTTA
Follistatin 2 (FS2) forward:
TGCCGAATGAACAAGAAGAA
reverse:
CAGAAAACATCCCGACAGGT

Each experiment described and its primers are but one incarnation and any primer consisting of at least 4 bases on each side of the Msp I or Fnu 4HI polymorphic sites can be employed to amplify the intervening genetic sequence, which can then be subjected to the restriction enzymes listed or other such enzyme and examined for the presence of either of the markers. The primers, enzymes and methods disclosed are in no manner meant to limit this invention in the detection of markers in follistatin that show some form of correlation with first parity litter size traits including number fully formed, number born alive and number of mummified fetuses.

Optimizing Primers: Twenty-four PCR reactions were conducted using a pool of DNA composed of ten or more individuals. At least 25 nano grams/micro liter of DNA was used per reaction along with 1×Taq reaction buffer (with MgCl₂), 1 micro liter DNTPs (100 mM), 0.5 micro liters of each primer (0.1 micro gram/micro liter), 1 unit of Taq and sterile water to bring reaction volume up to 25 micro liters. Two sets of twelve reactions were run through a thermal profile including a temperature gradient of 50° C.-65° C. to determine optimal annealing temperatures. The thermal profile was as follows: 96° C. for 10 min, 95° C. for 30 sec, Temperature gradient 50° C.-65° C. for 30 sec, 70° C. for 30 sec, Repeat steps 2-4 for 35 cycles, 70° C. for 10 min. Products from PCR were visualized using a 1% agarose gel with ethidium bromide to determine which temperature yielded the best product.

### EXAMPLE 4

PCR amplification: PCR was conducted using the reaction mixture described previously in example 3. Cycling conditions were: 96° C. for 10 min, 95° C. for 30 sec, Appropriate annealing temperature for the marker for 30 sec, 70° C. for 30 sec, Repeat steps 2-4 for 35 cycles, 70° C. for 10 min. Follistatin 1 PCR was carried out with an annealing temperature 56° C., while Follistatin 2 PCR was performed with an annealing temperature of 62° C. Temperatures and cycling conditions listed are those which produced optimal PCR product. Other conditions also worked.

### EXAMPLE 5

Restriction Digest Trials as a search for RFLPs: A DNA pool for each PCR product was digested with a panel of restriction enzymes to identify cutters. Manufacturer's temperature recommendations were followed for each enzyme. Twenty micro liter digests were conducted using 10 micro liters of PCR product, 7.7 micro liters of sterile water, 2 micro liters of buffer supplied with the enzyme, 0.2 micro liters BSA and 0.1 micro liters of enzyme. Digests were allowed to incubate at the enzyme's optimal temperature for three hours. Digest results were visualized using a 3% agarose gel with ethidium bromide. Digest results were run alongside uncut PCR product to determine if the enzyme cut effectively.

All enzymes deemed to be cutters were further tested to see if they would produce polymorphisms. This was done by using PCR from 24 individuals. Products from these individuals were then digested by each enzyme deemed to be a cutter for that particular PCR product. Digest results were visualized on a 3% agarose gel with ethidium bromide to see if resulting banding patterns differed among individuals tested.

Restriction Fragment Length Polymorphisms were discovered in the two amplified products of follistatin. The enzymes and polymorphic band patterns are listed in Table 1.

TABLE 1

Primer sequences, restriction enzymes,
product sizes of each marker

| | | | | | |
|---|---|---|---|---|---|
| Genetic | Primer Sequence | Restriction | A | B | Uncut |
| Marker | (5′ to 3′) | Enzyme | (bp) | (bp) | (bp) |

| | | | | | |
|---|---|---|---|---|---|
| FS1 | GGACCGAGGAGGACGTAAAT | Msp I | 220 | 425 | 625 |
| | GGCCTTTCCAGGTGATGTTA | | 225 | | |
| FS2 | TGCCGAATGAACAAGAAGAA | Fnu 4HI | 200 | 125 | 450 |
| | CAGAAAACATCCCGACAGGT | | | 75 | |

### EXAMPLE 6

Genotyping Generation Ten Individuals for Both Markers: To determine if these two RFLPs were worth pursuing, gilts from generation ten of the populations described in example 1 were genotyped. Samples were collected and DNA was extracted as described in examples 1 and 2. Amplification and restriction digestion using the conditions described in example 4 and Table 1 were carried out on 31 control line and 68 select line animals.

Genotyping: Following digestion, bands were visualized using a 3% agarose gel with ethidium bromide. Individuals were genotyped, and results were recorded. A couple of weeks later gel images were again scored to check for genotyping accuracy. Questionable genotypes were amplified again. Allele frequencies were calculated for the control and select lines. The allele frequencies were as follows for the B allele: FS1—control line=0.339, select line=0.588 and FS2—control line=0.489, select line=0.577. Because the differences in allele frequencies between the two lines seemed somewhat large considering the lines had only been divided for ten generations, it was decided that the RFLPs were worth pursuing.

### EXAMPLE 7

Genotyping Generation 11: The methods of example 7 were followed for generation 11 individuals. However, because in all but 2 instances the FS2 test yielded the same results as the FS1 test, only FS1 was used. In generation 11, 48 control line and 139 select line animals were tested. Again allele frequencies were calculated for both lines: FS1—control line=0.344, select line=0.529.

### EXAMPLE 8

Statistical Analysis: Various statistical analyses were conducted using a compilation of generation ten and eleven data in order to realize any trends.

Allele frequencies from examples 7 and 8 are presented in Table 2.

TABLE 2
Allele frequencies overall and within control and select lines

| Marker | | | | |
|---|---|---|---|---|
| (generation) | Allele | Overall | Control | Select |
| FS1 (10) | A | 0.49 | 0.661 | 0.412 |
| (n = 99) | B | 0.51 | 0.339 | 0.588 |
| FS1 (11) | A | 0.519 | 0.656 | 0.471 |
| (n = 187) | B | 0.481 | 0.344 | 0.529 |
| FS2 (10) | A | 0.511 | 0.7 | 0.423 |
| (n = 95) | B | 0.489 | 0.3 | 0.577 |

Breeding values for number of fully formed fetuses (NFF), number born alive (NBA), and mummified fetuses (MUM) in generations ten and eleven were estimated for each individual using an animal model in MTDF-REML. A summary of breeding value data for litter traits for both generations examined for each line is presented in Table 3.

TABLE 3
Mean breeding values for litter traits and
inbreeding values by line and generation.

| Trait | | Generation | Standard | Generation | Standard |
|---|---|---|---|---|---|
| Measured | line | 10 | deviation | 11 | deviation |
| NFF | c | −0.0464 | 0.25 | −0.032 | 0.203 |
| | s | 0.498 | 0.345 | 0.619 | 0.213 |
| NBA | c | −0.0315 | 0.189 | −0.018 | 0.153 |
| | s | 0.44 | 0.309 | 0.546 | 0.197 |
| MUM | c | 3.35* | 8.69* | 5.48* | 10.7* |
| *×10⁻⁸ | s | 1.28* | 4.26* | 1.48* | 5.09* |
| Inbreeding | c | 0.136 | 0.025 | 0.174 | 0.0197 |
| Coefficient | s | 0.181 | 0.0235 | 0.206 | 0.0292 |

Raw phenotypic litter data was recorded for each female genotyped in generations ten and eleven. The averages for these traits are presented in Table 4.

TABLE 4
Average phenotypic values for litter traits and
inbreeding values by line and generation.

| Trait Measured | Control/Select | Generation 10 | Generation 11 |
|---|---|---|---|
| NFF | c | 9.666667 | 8.363636 |
| | s | 9.886792 | 9.193548 |
| NSB | c | 0.291667 | 0.333333 |
| | s | 0.226415 | 0.408602 |
| NBA | c | 9.375 | 8.030303 |
| | s | 9.660377 | 8.784946 |
| MUM | c | 0 | 0.121212 |
| | s | 0.018868 | 0.086022 |

A standard chi-square analysis was done to determine possible deviations from Hardy-Weinberg equilibrium. All markers were tested for Hardy-Weinberg equilibrium. Results of those tests can be found in Table 5. Markers were not expected to be in Hardy-Weinberg equilibrium due to violation of the assumptions of no selection and no genetic drift. The select line has undergone several generations of direct selection. The other infringement on the Hardy-Weinberg assumption of no random drift is violated in both lines due to the small population sizes. It is impossible to avoid random genetic drift in small populations. Deviations from an ideal population in Hardy-Weinberg equilibrium were not significant for any markers in the control line. However, the deviation was significant for FS1 in the select line in generation eleven.

TABLE 5
Observed numbers of genotypes in control and
select lines, chi square values for Hardy-Weinberg
Equilibrium and associated P values.

| Gene | | | | | | | |
|---|---|---|---|---|---|---|---|
| (gener- | | | | | | | |
| ation) | Genotype | Control | chi² | P value | Select | chi² | P value |
| FS1 | AA | 12 | 1.56 | 0.212 | 10 | 0.586 | 0.444 |
| (10) | AB | 17 | | | 36 | | |
| | BB | 2 | | | 22 | | |
| FS1 | AA | 20 | 0.19 | 0.667 | 33 | 6.63 | 0.01 |
| (11) | AB | 23 | | | 65 | | |
| | BB | 5 | | | 41 | | |
| FS2 | AA | 13 | 2.18 | 0.139 | 9 | 1.79 | 0.181 |
| (10) | AB | 16 | | | 37 | | |
| | BB | 1 | | | 19 | | |

Changes in allele frequency were determined by difference between the control and select lines. A 2×2 contingency table was used to test for significant allele frequency differences between lines for FS markers. All differences were found to be significantly different from zero (FS1 P=0.0335, FS2 P=0.00165). A variance was calculated for each difference in gene frequency using formula 1 listed below, where p and q are allele frequencies, s and c are lines, g is generation and N is number of individuals with genotypic data per line. Variances for allele frequency differences that accounted for drift were also calculated using formula 2 listed below. In this formula F represents average inbreeding in that generation. Variances were converted to standard errors by taking the square root of each variance. Allele frequency differences along with the two measures of standard error are presented in Table 6.

$$\begin{matrix}{V}_{{p}_{s} - {p}_{c}} = \frac{{p}_{gs} {q}_{gs}}{2 {N}_{gs}} + \frac{{p}_{gc} + {q}_{gc}}{2 {N}_{gc}} & Formula 1 \\ {V}_{{p}_{s} - {p}_{c}} = {p}_{gc} {q}_{gc} ( 1 / [ 2 {N}_{gc} ] + {F}_{gc} ) + {p}_{gs} {q}_{gs} ( 1 / [ 2 {N}_{gs} ] + {F}_{gs} ) & Formula 2\end{matrix}$$

When attempting to account for drift, by incorporating inbreeding coefficients into variances of allele frequency differences among lines, standard error estimates increased greatly and thus, differences were non-significant. When using populations that are so highly inbred, it is difficult to attribute statistically significant differences to a particular gene because it stands to reason that highly inbred individuals share numerous genes which may in combination cause them to have similar litter traits.

TABLE 6
Allele frequency differences of the select line from the control

| Genetic Marker | Generation | Select-Control | SE w/drift | SE w/o drift |
|---|---|---|---|---|
| FS1 | 10 | 0.25 | 0.288 | 0.0134 |
| | 11 | 0.185 | 0.307 | 0.057 |
| FS2 | 10 | 0.277 | 0.279 | 0.0663 |

Additive (a) and dominance (d) effects were calculated using estimate statements with orthogonal contrasts of solutions for genotypic effects. Additive effect is defined as half the difference between Least Squared (LS) means of the two homozygotes. Dominance effect is the heterozygote LS mean minus the average of the two homozygote LS means. Additive and dominance effects are listed in Table 7. Follistatin 2 was not examined because this marker yielded the same genotype as FS1 in all but two pigs. Additive effects for all three traits were found to be significant or highly significant from zero for FS1 in the control line. These effects were found to be negative for NFF and NBA. This means that the favorable allele is different in the control line than that of the select line. When examining MUM, however, the favorable allele is like that seen in the select line. Additive effects for the B allele were found to significantly differ from zero in the select line for NBA.

TABLE 7
Additive (a) effects of favorable allele and dominance (d)
effects for breeding values of litter traits for FS1.

| Additive/ | | | | | | | |
|---|---|---|---|---|---|---|---|
| Domi- | Select/ | | | | | MUM | SE |
| nance | control | NFF | SE | NBA | SE | (×10⁻⁸) | (×10⁻⁸) |
| a | C | −0.3* | 0.10 | −0.20* | 0.09 | −10** | 2.5 |
| d | C | 0.02 | 0.06 | 0.01 | 0.06 | −1.0 | 1.6 |
| a | S | 0.061 | 0.05 | 0.09* | 0.04 | −1.3 | 1.23 |
| d | S | −0.007 | 0.03 | −0.01 | 0.03 | −0.3 | 0.86 |

*P value < 0.05
**P value < 0.0001

Data were analyzed using PROC GLM (SAS Inst., Inc., Cary, N.C.). The model included fixed effects of farrowing season, line, genotype and genotype*line. Dependent variables were breeding values for NFF, NBA and MUM in generations ten and eleven, which were estimated using an animal model in MTDF-REML. When examining follistatin markers, the genotype*line interaction term was found to be significant in the model for breeding values of NFF, NBA and MUM (P=0.0047, 0.0088, <0.0001, respectively). In the same models, genotype tended to be significant for NFF and was significant for MUM (P=0.0941, <0.0001, respectively).

Least square (LS) means were calculated for each trait and are presented in Table 8. For FS1, the favorable genotype appears to be line dependent, with BB being favorable in the select line and the opposite being true in the control line. When looking at pair wise differences of LS means for each genotype by line, AA differed significantly from both AB and BB in the control line (P=0.0251, 0.0037, respectively), and AB tended to differ from BB (P=0.0951) for NFF breeding values. In the case of NBA breeding values, AA tended to differ from AB (P=0.08) and differed from BB (P=0.028). In the select line for this same trait, BB differed from AA (P=0.03) and tended to differ from AB (P=0.09). When comparing genotypes in the control line for MUM breeding values, AA was found to be significantly different from AB and BB (P=<0.0001, <0.0001). The BB genotype was also found to differ from AB (P=0.0396). It is odd that in the control line for NBA, the heterozygote is more like the AA homozygote, while it is the opposite in the select line. This is probably due to the low occurrence of BB individuals in the control line.

TABLE 8
LS means for litter trait breeding values for FS1

| Control/ | | | | | | MUM | SE |
|---|---|---|---|---|---|---|---|
| select | Genotype | NFF | SE | NBA | SE | (×10⁻⁸) | (×10⁻⁸) |
| c | AA | −0.02 | 0.05 | −0.03 | 0.04 | 9.26 | 1.1 |
| | AB | −0.15 | 0.04 | −0.12 | 0.04 | 2.1 | 1.0 |
| | BB | −0.31 | 0.09 | −0.23 | 0.08 | −3.1 | 2.3 |
| s | AA | 0.47 | 0.04 | 0.38 | 0.03 | 1.91 | 0.93 |
| | AB | 0.49 | 0.03 | 0.42 | 0.02 | 1.57 | 0.63 |
| | BB | 0.53 | 0.03 | 0.48 | 0.03 | 0.63 | 0.79 |

When analyzing the data by line, FS genotype was found to be significant for NFF, NBA, and MUM breeding values in the control line. Follistatin genotype tended to be significant for NBA breeding value in the select line. This analysis revealed the AA genotype to be most favorable in the control line for NFF and NBA, while BB was found to be most favorable in the select line for the same traits. This result could be due to the small numbers of BB individuals in the control line. It could also be due to differing epistatic effects between the two lines.

Discussion and Conclusions: A candidate gene approach has been engaged to locate a major gene for economically important traits in the livestock industry. Rothschild et al. proposed the estrogen receptor gene as a gene associated with a major gene of litter size, incorporated previously. The present inventor has examined the follistatin gene as a candidate gene to investigate its effect on pig litter size. Differences for FS in frequencies between the two crossbred lines were anywhere from approximately 0.19 to 0.28 depending on marker and generation. Early indications show that there is a tendency for the follistatin markers to be associated with first parity litter traits in gilts.

### EXAMPLE 9

Sequencing PCR products: The PCR products were cleaned using a Qiagen (Valencia, Calif.) clean up kit. Clean PCR products were run through a sequencing PCR protocol using big dye. These PCR products were then sent to the Genomics Research Lab at North Carolina State University for sequencing to verify that the product was indeed a portion of the follistatin gene and to verify genotypes. Sequence data were analyzed using two computer programs—Consed and Polyphred (University of Washington). Each sequence was examined for good quality scores. Sequences were inspected for single nucleotide polymorphisms (SNPs).

### EQUIVALENTS

Persons skilled in the art will identify, or be able to determine through routine experiment, other assays and methods to test for the follistatin genetic markers described in this disclosure. Such other methods are intended to be encompassed by this disclosure.

What is claimed is:

1. Two different genetic mutations within the porcine follistatin gene, which have been found to be associated with litter size in first parity gilts.

2. A method comprised of obtaining a sample of genetic material from a pig and analyzing the genomic material to determine which follistatin (FS) allele(s) is/are present in said sample to screen pigs in order to determine those more likely to produce larger first parity litters and produce offspring also possessing that same ability.

3. A method as claimed in claim 2 wherein the determination of FS alleles in the analysis step encompasses determining the presence of at least one allele associated with at least one polymorphism in FS.

4. A method as claimed in claim 3 wherein the DNA markers are single nucleotide polymorphisms.

5. A method as claimed in claim 4 wherein the DNA marker is FS1 or FS2.

6. The method of claim 2 wherein said method of identifying the presence or absence of a polymorphism is selected from a group consisting of: restriction fragment length polymorphism (RFLP) analysis, heteroduplex analysis, single strand conformational polymorphism (SSCP), denaturing gradient gel electrophoresis (DGGE), temperature gradient gel electrophoresis (TGGE), DNA sequencing or other similar technique that can be utilized to detect differences in sequence between DNA strands.

7. The preferred method of claim 2 wherein said step of assaying for the presence of said polymorphism comprises the steps of digesting genetic material with a restriction enzyme that cleaves the pig follistatin gene in at least one place, separating the resulting fragments, detecting a restriction pattern, and comparing the pattern with a second restriction pattern for the follistatin gene obtained using the same enzyme, wherein the second restriction pattern is associated with increased first parity litter size.

8. The method of claim 7 wherein said restriction enzyme is MspI or other such restriction endonuclease that will differentially cut at FS1 polymorphism.

9. The method of claim 7 wherein said restriction enzyme is Fnu4HI, or other such restriction endonuclease that will differentially cut at FS2 polymorphism.

10. The method of claim 7 wherein said separation is by gel electrophoresis.

11. The method of claim 7 wherein said step of comparing said restriction patterns comprises identifying specific fragments by size and comparing the sizes of said fragments.

12. The method of claim 7 further comprising the step of amplifying the pig follistatin gene or a portion thereof, which contains said polymorphism, prior to the digestion step.

13. The method of claim 7 wherein said polymorphism is a polymorphic MspI restriction site if said amplification is of the region including exons 2 and 3 as well as the sequence between in the pig follistatin gene.

14. The of claim 7 wherein said polymorphism is a polymorphic Fnu4HI restriction site if said amplification is of the region including exons 3 and 4 as well as the sequence between in the pig follistatin gene.

15. The method of claim 12 wherein said pig follistatin gene is located on chromosome 16.

16. The method of claim 7 wherein said amplification is of the sequence spanning from exon 2 through exon 3 or the sequence from exon 3 through exon 4 includes selecting a forward and reverse sequence primer capable of amplifying a region pig follistatin gene which contains a polymorphic MspI site or Fnu4HI site or other appropriate restriction site.

17. The method of claim 16 wherein said forward and reverse primer sets amplify the region on chromosome 16 associated with the pig follistatin gene.

18. The method of claim 16 wherein said forward and reverse primers amplify a polymorphism found in the sequence of the follistatin gene, especially if that region covers the sequence from exon 2 through exon 3 or exon 3 through exon 4.

19. The method of claim 16 wherein said primers are SEQ ID NO: 1 and SEQ ID NO: 2 for FS1 marker and SEQ ID NO: 3 and SEQ ID NO: 4 for FS2 marker.

20. The method for identifying a polymorphism for pig first parity litter size comprising the steps of: (i) determining the litter size of each first parity gilt, (ii) determining the polymorphism in the follistatin gene of each pig wherein the polymorphism is identifiable by amplification by a set of primers selected from the group consisting of the set of a forward primer SEQ ID NO: 1 and reverse primer SEQ ID NO: 2 or the set of a forward primer SEQ ID NO: 3 and reverse primer SEQ ID NO: 4, and (iii) associating the litter size of each pig with said polymorphism thereby identifying a polymorphism for first parity litter size on pigs.

21. The method of claim 20 further comprising selecting pigs for breeding which are predicted to have larger first parity litter sizes by said marker.

22. The method of claim 20 wherein said analysis comprises digestion of PCR amplified DNA with the restriction enzyme MspI if said amplification is of the region from exon 2 through exon 3 or Fnu4HI if said amplification is from exon 3 through exon 4.

23. The method of claim 20 wherein said polymorphism associated with larger first parity litter size is amplified by use of forward and reverse primers comprising at least 4 consecutive bases in SEQ ID NOS: 1 and 2 or 3 and 4.

24. The method for determining the presence of a polymorphic site in the follistatin gene which is associated with an increase in first parity litter size in pigs comprising: (i) obtaining genetic samples from male and female pigs of the same breed, or breed cross, or derived from similar genetic lineages grown under normal conditions, (ii) determining reproductive traits of each pig from which a genetic sample was obtained, (iii) analyzing the genetic samples for polymorphisms in a gene associated with increased first parity litter size wherein the gene is the follistatin gene wherein the polymorphisms are identifiable by amplification by a set of primers selected from the group consisting of the set of a forward primer SEQ ID NO: 1 and reverse primer SEQ ID NO: 2 or the set of a forward primer SEQ ID NO: 3 and reverse primer SEQ ID NO: 4 or a comparable set of primers, (iv) performing an appropriate restriction digest or similar analysis to determine which FS alleles are present, and (v) correlating the polymorphism(s) to larger first parity litter size by comparing the presence of polymorphisms to litter records.


SEQUENCE LISTING (7 sequences)

SEQ ID NO: 1 - DNA, 20 bp, Sus scrofa
ggaccgaggaggacgtaaat

SEQ ID NO: 2 - DNA, 20 bp, Sus scrofa
ggcctttccaggtgatgtta

SEQ ID NO: 3 - DNA, 20 bp, Sus scrofa
tgccgaatgaacaagaagaa

SEQ ID NO: 4 - DNA, 20 bp, Sus scrofa
cagaaaacatcccgacaggt

SEQ ID NO: 5 - DNA, 631 bp, Sus scrofa
ggaccgaggaggacgtaaatgacaacacacttttcaagtggatgattttcaatggaggtgcccccaactgcatcccatgtaaaggtaagggtcttcctccccaatttgcaggccctcagtagaggggtcttaccctcagcttccccactgcccagctggcgtttgtttgagcctgggagagctttgttggtgggttccccctaactgcctccagccccagtaagccattgggcgttgaattttggcagtcgtataccacaggcaaaacaagatctgcagaggttcgctgacctcttagccaaatgcggtggtgacaccagccagccctctcactttgagagagatttgggtatgggaacacaaatctgtcccccacccctcctcaatttctgggcgagagcctagacccctcaggcttgaactcccggctcgcgattgcgcaaggcacccgaagccctcctggctgacctgtttggtgcctggccctggttttaatcccctgcctctttccaactcttagaaacgtgcgagaacgtggactgtgggcccgggaaaaaatgccgaatgaacaagaagaacaaaccccgctgcgtctgcgccccggattgttctaacatcacctggaaaggcc

SEQ ID NO: 6 - DNA, 504 bp, Sus scrofa
tgccgaatgaacaagaagaacaaaccccgctgcgtctgcgccccggattgttctaacatcacctggaaaggcccagtctgtgggctggatgggaaaacctaccgcaacgaatgtgctctcctcaaggccagatgtaaagagcagccggaactggaagtccagtaccaaggcaaatgtaaaagtaggtcacccctcctccagctttcaacctgaggtagtcccgcagcaagacccctggggtttggtgtagtcacagtaagagcctaatgatatcaaataaaggaacccttttctaatgactccttaagaacgctgaaggcaacccagaggcacagggtttttttttttgaaaaccacagcgttcccaaaataaatttttgaaagctggatgcttccattcatgatttccttgatagtatcaaatgctgagtcccaaagtacaggaaggtgcctattaacgtgtgtttctctctttgtttcagagacctgtcgggatgttttctg

SEQ ID NO: 7 - DNA, 6247 bp, Sus scrofa
tgtcccgcgctaggttacaggggcctaaacaccagcgacagacccggagtggggtgtttcagcagctcggggatcgccgggcatttgcaaaagtcttcttggagcggagcggcgatgcgaagaccaattccgcccgctctcgtccgccagccgccacccccagcccaccatgccccccccaacgcacacactcacacaccaccatgaacgaacacacacacacacacacacacacacgccgtcccaggcgcgccggcgctgcctgcgagcggcggcgtgcaggacttgaagtgggtgttcttccccactccccacccccgaagcgtcgccctcacccccctccgtcccggtcgcctgccccctccctctcgctcctacgcaaataagaactcggcgataccctcctgccgctttgatttcgggcacctccaccagataattgggaagggtttccagaaggtgggaaatgtcacctgattcacactgaacttttaaaagctccccacccccaaggagccgcgcacaccctcgctcgaggccgccctcccactgccccacacactgggagaccgcccaccgcaaaccgcggagacccccgtctagatttaaagcgcggctgcgcccggcttctgacgtccattgaatcgcgcgggcggccggcggcgagcgcggggttgcgccgggatcgctgcgccctccgccgctcgcctctgcgacgcgcgccgctcgccggaaccactcgccgccgcgcctgctccccgcaccgtcgcgctcctcgtcccgcgcctgaccccagaatggtccgtcccaagcaccagcccggcgggctatgcctcctgctgctgctgctctgccagttcatggaggaccgcagcgcccagggtaagagaagggggatgcgctgggaggctcggcaggcgacaggggtttgtacttttcagtcgtctccactggtctgttctgagccctgctcttagatgttggaccaacttaggaagtccggatggagaggagtgggaaaattgaaggcagtgacactttaggactgggagccgagtttggagcggggcgatcctttaaaaggaaagggagtgagctggagtctgtctcagtagttagggttagatgctccaagttctggacataatgagtttagatgttatctcccctgtttggctctgaagaaggggaaaagcatcagtagcctggagatttctgtcgacccacttacagtctacctctctacctcctacatcttttggatactttaaaaaatgactgcattggggagggggatactgaaatttcttcggtggctgagggacgtggtttcagaaactttgcccaaacttggaaatgtgggtgccgaaaattttagagggaagtagctttctgcctagtggatgcttcttccaacaccctcccccaaacccgcaaaaaaaaaaaaaaaaaaaaaaaaatacccctgactgttttcagtagtttacaattttttttctttgcctcattcctcatgttaactttctgtagaacagtgaaaagcccacaattgatttaaattctgagatgcagtaagagcaaagtatttttttaattagatttatttatttctgctagtctttgggcagacttttttaaaatcataaaagcagcttgctgttcaatatattgttaagattatagggatgattcccgagaggtctttctgaccaaatccgaacattaaaaaggaggagggggggggaattacttgcaaagatttcttggtaggtatgagatatgtattaccatgagacggtggaagcccctcccccctttattattattatttacttggttggttgggttttttccccctacactcttcttgagacggacttgtaactttcctggctttagcattaacattttatgtgtgttgtaaactggccaccgggcactgcccctcgcggtctgcaacgcaccactctgagttgcccgcctttcggggcgaggcgctgcctaggtgccggggacgtgggcgccggggaaagcagccctcggagatttcgaaagatcgcggtggccgcgggcgtcctctggatctggcggattgcaaaacgggaccgtcgggagggctgcgcttgaggccgcacggggcagctccgggcaaggctcagttgcttttgcttttttgctttcatttttctcaagtgccttttgcttgatctgttcctgtaggtgtctgtctcagcctctccaccctcccccccttttttctttctcttaagttctcctactttcttaacccctgtcacaaataatacttttaccgcccacatgaaaccaacatgtaaaaacatccgtcgcatcctgtttttgtcaggttctttaatctgctcttcgagtcgctgcaggttatgaaataggacgaataaaagtaaacagtctagtaaaagtcaatgcaagctgcacgtgttgtgtctgggtcactggtaactgacattgatatggctggggcgctctggcttctcgctctcgccctccctccccaccgccccctcgcccacctcccacttcttcaatctggctccccccctttacacaccccgtcattcccacctatttattttctgcttttctcttctctctcgcctcctcctccacgctcactccctccccatccccgaagggtttccttgcctcgaaagctcaccgcctccctacccgaacccctccgctcacttgcccacctccccacccctgtctccccgcagctgggaattgctggctccgccaagcaaaaaacggccgctgccaggtcctgtataaaaccgaactgagcaaggaggagtgctgcagcaccggccgcctgagcacctcctggaccgaggaggacgtaaatgacaacacacttttcaagtggatgattttcaatggaggtgcccccaactgcatcccatgtaaaggtaagggtcttcctccccaatttgcaggccctcagtagaggggtcttaccctcagcttccccactgcccagctggcgtttgtttgagcctgggagagctttgttggtgggttccccctaactgcctccagccccagtaagccattgggcgttgaattttggcagtcgtataccacaggcaaaacaagatctgcagaggttcgctgacctcttagccaaatgcggtggtgacaccagccagccctctcactttgagagagatttgggtatgggaacacaaatctgtcccccacccctcctcaatttctgggcgagagcctagacccctcaggcttgaactcccggctcgcgattgcgcaaggcacccgaagccctcctggctgacctgtttggtgcctggccctggttttaatcccctgcctctttccaactcttagaaacgtgcgagaacgtggactgtgggcccgggaaaaaatgccgaatgaacaagaagaacaaaccccgctgcgtctgcgccccggattgttctaacatcacctggaaaggcccagtctgtgggctggatgggaaaacctaccgcaacgaatgtgctctcctcaaggccagatgtaaagagcagccggaactggaagtccagtaccaaggcaaatgtaaaagtaggtcacccctcctccagctttcaacctgaggtagtcccgcagcaagacccctggggtttggtgtagtcacagtaagagcctaatgatatcaaataaaggaacccttttctaatgactccttaagaacgctgaaggcaacccagaggcacagggtttttttttttgaaaaccacagcgttcccaaaataaatttttgaaagctggatgcttccattcatgatttccttgatagtatcaaatgctgagtcccaaagtacaggaaggtgcctattaacgtgtgtttctctctttgtttcagagacctgtcgggatgttttctgtccaggcagctccacatgtgtggtggaccagactaataatgcctactgtgtgacatgtaaccgcatttgcccagagcccacctcctcagaacagtatctctgtgggaatgatggagtgacctactccagtgcctgtcacctgagaaaggctacctgcctactgggcagatctattggattggcctatgagggaaagtgtatcagtaggtattctggattgagaaaggaaaagaaataaatgggaaaagggaaaaaggctaattctgtcattaaaaaaagcctaaagcctccccaaacacatagcttcagcaaagggagaaatattctccagtttgggtaaactgtcctgctcacagcactgtcttccagaagcatcagcacatatattgaaatggcagcaagacacaggaaacaattttccttcatagaaacccaagacctcactcacttttacacatcttttaaagtgctagacttgctggaagcaagaaataattacttaacagttcctaaaatctgttctcagattctgataactaatggaacttcttttttttaagatcccacttatgggaaataaataaatatgtatatttaactcacagatattgtgaaatcctgttcccccaaaaaaactacagtattttcatgcactaattatagtatgaagggtgtgtgtgtgtgtgttgagtttcaccggttttttaaaagaacatttttgcaccatatctccattaacccccttaatgagtaataaggtatgttatatgtttacatttattgataggagactatagagaaaagggggcaagggggaaatggatttactcatcacaggtgtattatatcctagaagcaaagtcctgtgaagacatccagtgcactggtggaaaaaagtgcttatgggatttcaaggttggcagaggccgctgttccctctgcgatgagctgtgccctgagagtaagtctgaggagcctgtctgtgccagtgacaatgccacctacgccagtgagtgtgccatgaaggaagcggcctgttcctcaggtgtgctgctggaagtaaagcactctggatcttgcaactgtaagtgtgatttctaacctttctgaaattgaaagctttcccaggcaggccctagggaatgcgatgtaaaaaagcacaaaaacctccccatataagcccagttaggtagctgctaagttttaccagcaattcagcaatccacagaaaaactgcctggaatttttcctgggcccttaggacttgtcaacgatcatcaatggggtcgcctctataaacctgtttccagtcattcacccctaatttctttagctttagtagtgtgctttgctttattaacacttgaatctaaactaactgcttcaaaggcgtttttttctttttcaaagacagctttctattatcatacatgggctgctgctttgcgcagttgcctctactaactctgaattaagaacccaaagcagttatacctagaacacgagcgctttttatctaatttcaggaatctgcccgtaaaacctgagccattgattcttcagaactttctgcagtttttgacttcatagataatgtttttaaaaaacattttttttaaacttaatgcataacagcagatgccaaaaagcaaaaaaaaaaccaaaaaaaaaaaaaagcatcttactgcaagtcacataaaaatgcaacgctgtaatatggctatttcagagggctttgaaaacatacactgagctgcttctgcgctgttgttgtccgtatttaaacagcagctcccctgtattcccccatctagccatttcagaagacaccgaggaggaggaggaagatgaagaccaggactacagctttcctatatcttctattctagagtggtaaaccctccaccaattttcagtgttgacatagcctttgggcaaaaaaaaaaaaaaaaaaaaaaaaagaaagaaaaaagaaaaaaaaagaaagaaagaaagagaaaaaagaaaaatatatattgtccatactgtaaataagtgtatgcttatttatttggggggaaaagtatacataaaggacctttgtcctaaagctctctcccaggccaccttgttactcattggacccggagaggcagtcattgtgaggtctactggatgaggcccatagttgagacttgtagacatttatttatactgtgtcatgttttataatttatacataaaatgtctggttgactgtacaccttgtttttgaagaaatttattcgtgaaaggaagagcagttgttatttattgtgaggtctcttgcttgtaaagtaaaa